(19) United States
(12) Patent Application Publication
(10) Pub. No.: US 2015/0025131 A1
(43) Pub. Date: Jan. 22, 2015

(54) PATULETIN A POTENT ANTI-TNF-a AND ANTI-ARTHRITIC COMPOUND FROM TAGETES PATULA

(71) Applicants: Muhammed Ahmed Mesaik, Karachi (PK); Almas Jabeen, Karachi (PK); Shaheen Faizi, Karachi (PK); Shabana Usman Simjee, Karachi (PK); Samina Bano, Karachi (PK); Lubna, Karachi (PK)

(72) Inventors: Muhammed Ahmed Mesaik, Karachi (PK); Almas Jabeen, Karachi (PK); Shaheen Faizi, Karachi (PK); Shabana Usman Simjee, Karachi (PK); Samina Bano, Karachi (PK); Lubna, Karachi (PK)

(21) Appl. No.: 13/944,387

(22) Filed: Jul. 17, 2013

(51) Int. Cl. A61K 31/352

(52) U.S. Cl. CPC A61K 31/352
USPC 514/456; 549/400

(57) ABSTRACT

Patuletin, a flavonoid isolated from Tagetes patula, was discovered to be a potent inhibitor of TNF-α production and thus useful in treating diseases related to TNF-α levels in the body.

### BACKGROUND OF THE INVENTION

Rheumatoid arthritis (RA) is most common chronic, inflammatory joint disease of worldwide distribution, causes irreversible joint destruction and functional impairment. It affects approximately 1% of the adult population with a female predominance of 3:1. Although the precise etiology still remains unclear, pathogenic mechanism involves intense inflammation in synovial joints, so that the normally delicate synovial membrane becomes infiltrated with mononuclear phagocytes, lymphocytes, and neutrophils. The course of RA is variable, but usually patients develop diminished mobility due to progressive loss of cartilage and bones around the joints.

In the early stages of inflammation, activated CD4⁺ T helper cells stimulate synovial fibroblasts, monocytes and macrophages to produce the proinflammatory cytokines TNF-α, interleukin (IL)-1β resulting in the secretion of degradative enzymes, called matrix metalloproteinases (MMPs). This further leads to cartilage erosions and destruction of bones and soft tissues. TNF-α is regarded as “master switch” cytokine in the inflammatory process and bone destruction.

Cytokine inhibition provides a feasible method for the treatment of chronic inflammatory diseases. Example includes the blocking of tumor necrosis factor (TNF-α) and interleukin (IL-1β) in RA. Inflammatory cells including macrophages, neutrophils, lymphocytes and endothelial cells produce reactive oxygen species (ROS) that involve both directly and indirectly in the progression of pathogenesis of inflammatory synovitis and oxidative damage. Although playing the central role in various physiological processes the increased production of NO is pathological. At the site of synovial inflammation it mediates the production of cytokines, signal transduction molecules and mitochondrial functions etc., playing an important role in the pathogenesis of RA. Antioxidant defense system includes enzymes like glutathione peroxidase, superoxide dismutase and catalase etc., have protective role against ROS, however, over production of ROS reduces the level of these enzymes leading to oxidative stress induced tissue damage in RA patients.

Tagetes patula (French marigold) is widely known for its phytochemical and medicinal properties. Traditionally the plant is used to treat cough, stomach disorders, and rheumatism. It is also known to possess antimicrobial, antiseptic, blood purifying, and diuretic, properties. The flowers of T. patula are edible and used in refreshing drinks. Chemically the different parts of T. patula were reported to contain triterpenes, steroids, flavonoids and thiophenes.

Flavonoids of different classes are known to possess several pharmacological and biochemical properties and also have a regulatory role on different hormones and have great therapeutic potential due to their wide biological actions. Patuletin was first isolated by, R. S. Rao and T. R. Seshadri in 1941 from the petals of T. patula and represented as 3,5,7,3′,4′-pentahydroxy-6-methoxy flavones, it belongs to a biologically active group of phenolic compounds “flavonols” that are widely distributed in nature. It is a non-toxic flavonoid and has also been reported from other Tagetes species. Patuletin is known to possess various biological activities, which include radical scavenging activities, anti-inflammatory using rat carrageenan model, antimicrobial, analgesic, antispasmodic, hypotensive, and nematicidal properties.

### BRIEF SUMMARY OF THE INVENTION

The invention reports the immunomodulatory potential particularly anti TNF-α and anti-arthritic activities of Patuletin with no cytotoxic property. The in vitro studies deal with the effect of this flavonoid on production of proinflammatory cytokines, ROS production, and proliferation of T-cells as well as its effect on P-38 MAPK and the transcription factor NFκB. The cytotoxicity of the compound was tested using three different cell lines by MTT assay.

The anti-arthritic potential of this compound was revealed by serological and histological studies using Adjuvant Induced Arthritis (AIA) model on Sprague Dawley rats. The serological studies include the effect of Patuletin on serum TNF-α, IL-1β, nitric oxide, RF (rheumatoid factor), and on the level of glutathione. Histologically it causes less destruction of bones, compared to the arthritic group, showing its anti-arthritic potential. Based on the in vitro and in vivo experimental studies, Patuletin could be consider as potential immunosuppressive, anti TNF-α and anti-arthritic lead candidate.

### BRIEF DESCRIPTION OF THE DRAWINGS

FIG. 1 depicts the effect of Patuletin on TNF-α production (pg/mL) Human monocytic leukemia THP-1 cells were differentiated and activated with 20 ng/mL of phorbol 12-myristate 13-acetate (PMA) and 50 ng/mL of LPS respectively and incubated with different concentrations of Patuletin for 4h, the supernatant was analyzed for the level of TNF-α by Enzyme linked immunosorbant assay (ELISA). The symbol * and δ represents the P≦0.03 and P≦0.006, respectively.

FIG. 2 depicts the effect of pentoxyfillin (PTX) on TNF-α production (pg/mL) Human monocytic leukemia THP-1 cells were differentiated and activated with 20 ng/mL of PMA and 50 ng/mL of LPS, respectively and incubated with different concentrations of PTX for 4h, the supernatant was analyzed for the level of TNF-α by ELISA. The symbol * represents P≦of 0.03.

FIG. 3 depicts the effect of Patuletin on signaling molecules and transcription factors. (a) P38 and Phosphorylated P38 (b) NFκB and Phosphorylated NFκB. PMA differentiated THP-1 cells were treated with 50 ng of LPS and three different concentrations (25, 8.3 and 2.7 μg/mL) of Patuletin for 4h. Total protein was subjected to 12% SDS-PAGE followed by western blotting. Equal amounts of protein were loaded according to protein estimation.

FIG. 4 depicts concentration of TNF-α (pg/mL) measured in the serum of normal, treated arthritic and non-treated arthritic rats. Values are given as mean±SEM for n=12 animals/group. The arthritic control group exhibited a significant increase in the level of TNF-α production in comparison to the normal control group. Whereas marked decrease was observed in the level of TNF-α in arthritic rats, treated with the Indomethacin, and Patuletin when compared to the arthritic control group. The symbols δ, τ and * represents P≦0.008, P≦0.009 and P≦0.006 respectively.

FIG. 5 depicts a concentration of IL-1β (pg/mL) measured in the serum of normal, treated arthritic and non-treated arthritic rats. Values are given as mean±SEM for n=12 animals/group. The arthritic control group exhibited a significant increase in the level IL-1β compared to the normal control group. There was a significant decrease observed in the level of IL-1β in indomethacin and Patuletin treated arthritic rats compared to the arthritic control group. The symbols δ and * represents the P≦0.01 and 0.003 respectively.

FIG. 6 depicts a concentration of Nitric oxide (μM) measured in the serum of normal, treated arthritic and non-treated arthritic rats. Each bar represents the mean±SEM for n=12 animals/group. The arthritic control group showed a marked increase in the level of NO production when compared to the normal control group. Whereas the treatment with the indomethacin, and Patuletin showed marked reduction in the level of NO when compared with the arthritic control group. * represents P≦0.001.

FIG. 7 depicts Serum Glutathione Level (mg/dl) measured in normal, treated arthritic and non-treated arthritic rats. Each bar represents the mean±SEM for n=12 animals/group. The arthritic control group showed a significant decrease in the level of glutathione when compared to the normal control group. The arthritic rats treated with indomethacin, and Patuletin showed non-significant increase in the level of glutathione when compared with the arthritic control group. * represents P≦0.001.

FIG. 8 depicts Serum Peroxide concentration (ng/ml) measured in normal, treated arthritic and non-treated arthritic rats. Each bar represents the mean±SEM for n=12 animals/group. The arthritic control group showed increase in the level of peroxide production when compared to the normal control group. The arthritic rats treated with indomethacin, and Patuletin showed decrease in the level of peroxide level when compared with the arthritic control group. The results are found to be statistically non-significant.

FIG. 9 depicts the level of RA factor (IU/mL) measured in the serum of normal, treated arthritic and non-treated arthritic rats. Values are given as mean±SEM for n=12 animals/group. The arthritic control group showed a significant increase in the level of RA factor in comparison to the normal control group. In contrast there was a marked decrease observed in the level of RA factor in arthritic rats treated with the indomethacin and Patuletin when compared with the arthritic control group. The symbols δ, τ and * represents P≦0.005, P≦0.007 and P≦0.003 respectively.

FIG. 10 depicts the effects of Patuletin on the histopathology of knee joints (H & E staining). (a) Normal control: articular bone is intact and few lymphocytes infiltration of synovium can be seen (score 0). (b) Arthritic control: a prominent lymphocytic infiltration of synovium with invasion of peri-articular bone and vacuolization, collapse of articular surface and articular bone destruction, and increase joint space can be seen (score 4). (c) Indomethacin treated group: compared to arthritic control, a reduction in inflammatory changes can be seen (score 2). (d) Patuletin treated group: reduction in the inflammatory changes observed (score 2). Where AC: articular cartilage; SM: synovial membrane; P: peri-articular tissue.

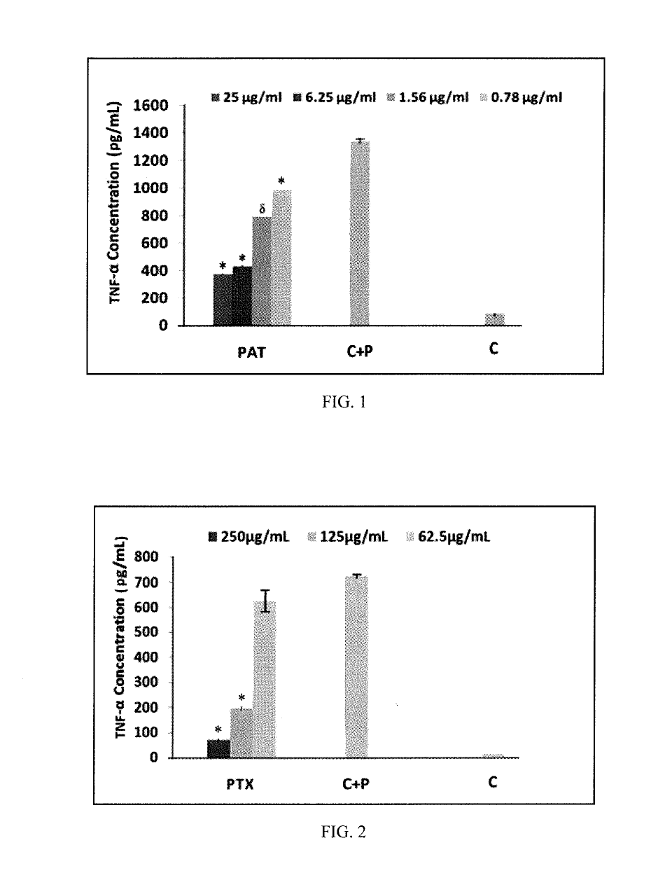

### DETAILED DESCRIPTION OF THE INVENTION

The compound Patuletin was tested for its effect on TNF-α and IL-1β production from the PMA differentiated and, LPS activated THP-1 cells. It strongly inhibits TNF-α production (IC₅₀=2.5 μg/mL). Results were compared with the pentoxyfillin (IC₅₀=94.8 μg/mL), a known TNF-α inhibitor. When tested for the inhibition of IL-1β production it shows moderate to weak inhibition (IC₅₀=>50 μg/mL).

Patuletin was found to possess no cytotoxicity, when tested on NIH 3T3 mouse fibroblast cells, and on MDBK bovine kidney cells, a moderate level of cytotoxicity was observed (IC₅₀=5.2 μg/ml) on CC1 cell line however, the effect was observed after 48 hours of incubation; whereas proinflammatory cytokine inhibitory effect was observed after 4 h and 18 h of incubation, respectively. Results were compared with the cyclohexamide, a standard cytotoxic drug.

Table 1. Shows the effect of Patuletin on proliferation of T-cells. The effect of Patuletin on PHA activated T cell, non-activated Jurkat cell proliferation and IL-2 production was analyzed. The IC₅₀ values were obtained using various concentrations of Patuletin and readings are presented as mean±SD of triplicates.

TABLE 1

| | Cytotoxicity (IC₅₀ μg/mL) | | |
|---|---|---|---|
| Compound | NIH 3T3 cells | CC1 cells | MDBK cells |
| Patuletin | 36.4 ± 6.9 | 5.2 ± 0.2 | 12.6 ± 0.40 |
| Cyclohexamide | 0.13 ± 0.02 | 0.02 ± 0.002 | 1.4 ± 0.06 |

The effect of Patuletin on extra and intracellular reactive oxygen species (ROS) production were studied. Intracellular effect was determined using human peripheral whole blood phagocytes and isolated neutrophils and from mice peritoneum macrophages, where luminol-enhanced chemillumenescence assay was applied. The effect on myeloperoxidase independent ROS production was determined using lucigenin on murine macrophages RAW.267 cells. The compound shows significant suppression on extracellular ROS production from whole blood (IC₅₀=3.5), and on isolated and serum opsonized activated phagocytic cells, with an IC₅₀ of 1.2 and 1.9 μg/mL on human neutrophils and on mice peritoneal macrophages respectively, results were compared to the Ibuprofen. The compound also found to potently inhibit extracellular ROS species (IC₅₀=0.3) compared to the DPI as standard drug.

Table 2. Present the effect of Patuletin on myeloperoxidase dependent and independent oxidative burst: (a) effect of Patuletin on Luminol enhanced oxidative burst on whole blood, zymosan activated PBMNs and mice peritoneal macrophages. Ibuprofen is used as standard drug and (b) effect of Patuletin on Lucigenin enhanced oxidative burst on PMA activated RAW. 267 cells (murine macrophages). DPI is used as standard drug. Reading presents mean±SD of three determinations.

TABLE 2

| | | | |
|---|---|---|---|
| Compound | Whole blood | Neutrophils | Macrophages |

(a) Myeloperoxidase dependent Oxidative Burst (IC₅₀ μg/mL)

| | | | |
|---|---|---|---|
| Patuletin | 3.5 ± 0.5 | 1.2 ± 0.2 | 1.9 ± 0.3 |
| Ibuprofen | 11.2 ± 1.9 | 2.5 ± 0.6 | 16.9 ± 2.5 |

(b) Myeloperoxidase independent Oxidative Burst (IC₅₀ μg/mL)
on macrophages

| | |
|---|---|
| Patuletin | 0.3 ± 0.03 |
| DPI | 1.6 ± 0.1 |

In order to monitor the effect of this compound on cellular immune response particularly on T cells, the compound was tested on thymidine incorporated T cell proliferation assay. The compound showed potent suppression on PHA activated T cells (IC₅₀=1.2 μg/mL) isolated from human peripheral blood, this activity was significantly comparable with prednisolone activity which gives IC₅₀=<0.62 μg/mL. It was also found to suppress the proliferation of Jurkat T cells activity resulting in an IC₅₀ of 3.47 μg/mL, While found to have a moderate inhibition on IL-2 cytokine production (IC₅₀=14.8 μg/mL).

Table-3. shows a toxicity evaluation of compound. The effect of Patuletin on viability of NIH 3T3 (mouse fibroblast), MDBK (bovine kidney) and CC1 (rat Liver) cell lines was analyzed by MTT assay. Cyclohexamide was used as a standard drug results are presented as mean±SD of three determinations.

TABLE 3

| | Human T cell | Jurkat T cell | |
|---|---|---|---|
| | proliferation | Proliferation | IL-2 inhibition |
| Compound | (IC₅₀ μg/mL) | (IC₅₀ μg /mL) | (IC₅₀ μg/mL) |
| Patuletin | 1.2 ± 0.1 | 3.47 ± 0.04 | 14.8 ± 1.8 |
| Prednisolone | <0.62 | — | — |

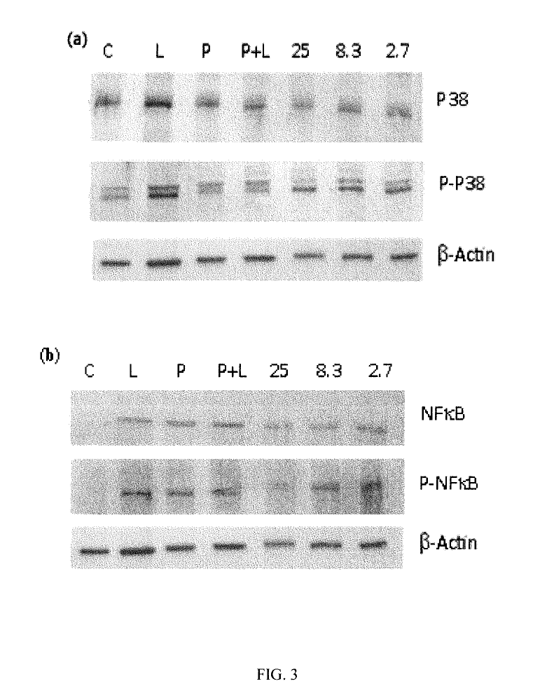

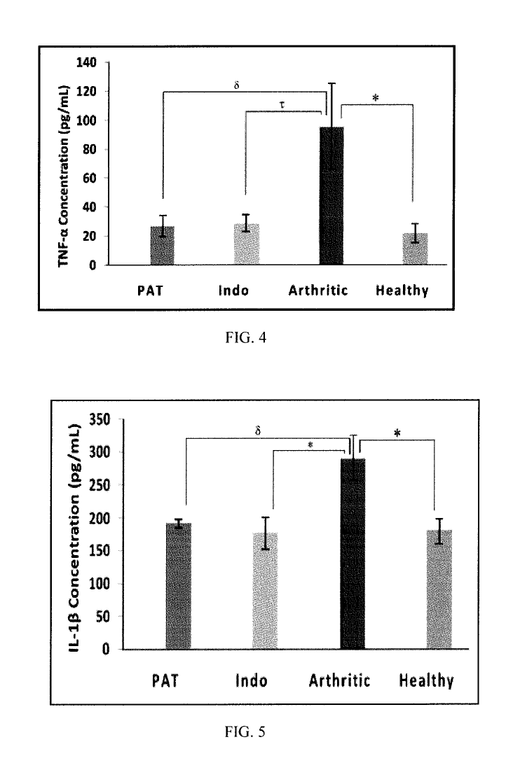

When tested for its effect on the expression of P38 MAP kinase and transcription factor NFκB along with their respective phosphorylated forms, from THP-1 cells, the Patuletin shows mild inhibition on p38 production at 25 μg/mL, inhibits NFκB and its phosphorylated form at all concentration whereas significant inhibition of NFκB phosphorylation was observed at concentration of 25 μg/mL [FIG. 3].

In vivo studies on the natural compound Patuletin was done using adjuvant induced arthritis model on female Sprague Dawley rats. Effects on various aspect of inflammation including inflammatory signals, serological and histological changes after induction of arthritis were analyzed and presented.

The effect of Patuletin on proinflammatory cytokines in the serum of normal, treated and non-treated arthritic rats was determined. The arthritic control group exhibited a significant increase in the level of TNF-α and IL-1β production in comparison to the normal control group. Whereas, a marked decrease in the level of TNF-α and IL-1β was observed in rats treated with Patuletin, and indomethacin, when compared with the arthritic control group.

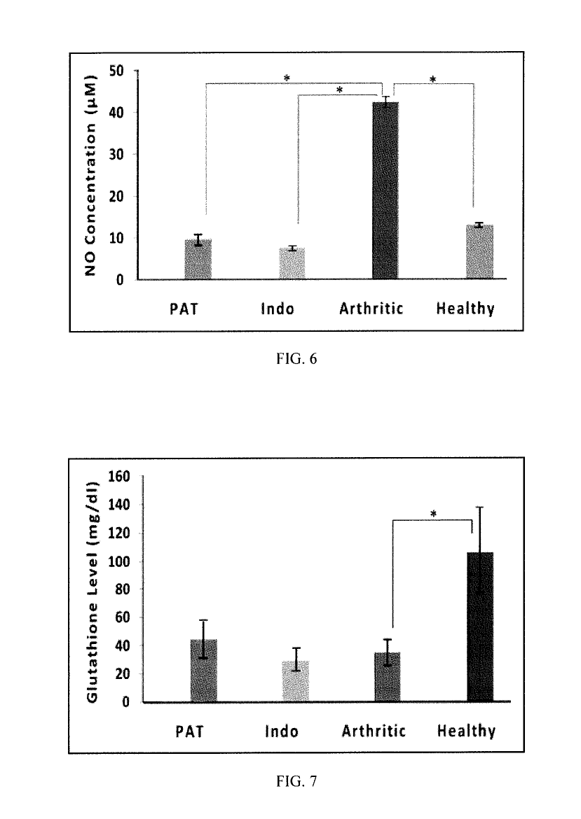

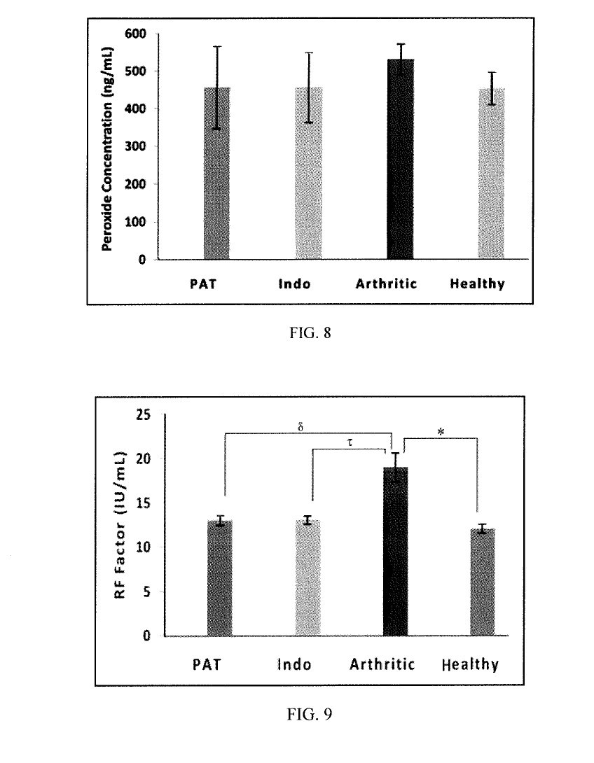

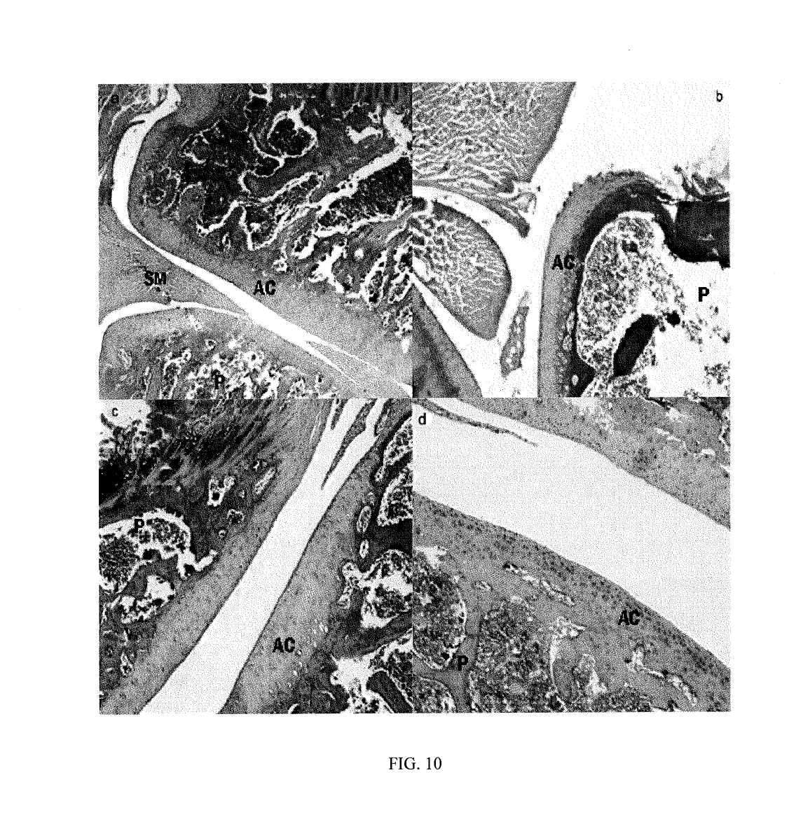

The compound was analyzed for its effect on inflammatory marker nitric oxide concentration in serum. Significant increase in the level of nitric oxide was observed in arthritic control group in comparison to the normal untreated group. Whereas the rats treated with the Patuletin and indomethacin, both showed significant decrease in the concentration of NO when compared with the arthritic control group [FIG. 6].

The Patuletin was tested for its effect on serum peroxide. The arthritic control group showed increase in the level of peroxide production when compared to the normal control rats, whereas non-significant reduction in the level of peroxide was observed in the serum of arthritic rats treated with Patuletin and indomethacin.

The level of glutathione was analyzed in the serum of normal, treated arthritic and non-treated arthritic animals. The arthritic control group showed a significant decrease in the level of glutathione when compared to the normal control group. The arthritic rats treated with Patuletin showed mild increase, whereas indomethacin treated rats showed decrease in the level of glutathione, compared to the arthritic control rats. However statistically this was found to be non-significant.

The level of (RF) rheumatoid factor was determined in the serum of normal, treated and non-treated arthritic rats. The arthritic control group exhibited significant increase in the level of Rheumatoid factor in comparison to the normal control group. In contrast there was a marked decrease observed in the level of rheumatoid factor in arthritic rats treated with the Patuletin and indomethacin, compared with the arthritic control rats.

Histopathalogy of knee joints from all experimental groups was performed. Intact articular bone and few lymphocytes infiltration were observed in normal control rats. All characteristic features of arthritis including proliferation of granulation tissue, lymphocytes infiltration, collapse of articular surface and cartilage destruction was observed in arthritic control rats. Rats treated with Patuletin and indomethacin shows less infiltration of lymphocytes and minimal bone destruction compared to the arthritic control rats.

THP-1 (human monocytic leukemia) cells were maintained in RPMI-1640 supplemented with 10% FBS, 2 mmol/L glutamine, 5.5 mmol/L glucose, 50 μmol/L mercaptoethanol, 1 mmol/L sodium pyruvate, and 10 mmol/L HEPES and incubated at 37° C. in 5% CO2. Cells were grown in 75 mm2 culture flask until they attained 70% confluency. Cells were then plated in 24-well tissue culture plates at a concentration of 2.5×10⁵ cells/mL. Cells were differentiated into macrophages using PMA at a final concentration of 20 ng/mL and further incubated at 37° C. in 5% CO2 environment for 24 h. Cells were then stimulated with bacterial lipopolysaccharide (LPS) 50 ng/mL, and treated with compound initially at concentration of 25 μg/mL to test their initial activity, then using five different concentrations (0.39, 0.78, 1.56, 6.25, 25 μg/mL) in order to calculate the IC₅₀. After addition of the compound cells were then incubated for 4 h for TNF-α production and 18 h for IL-1β production at 37° C. in 5% CO₂. The supernatants were harvested and analyzed for the level of TNF-α and IL-1β, by human TNF-α and IL-1β Duo set ELISA kits, and according to the manufacturer's instructions.

Heparinized blood was obtained by vein puncture aseptically, from healthy volunteers (25-38 years age). The buffy coat containing polymorhonuclear neutrophils (PMNs) was collected by dextran sedimentation, and cells were isolated after the LSM density gradient centrifugation. PMNs were collected from the tube base along with the red blood cells (RBCs). Cells were obtained after the RBCs lysis using hypotonic solution, and washed twice then suspended in Hank's balance salt solution without Ca⁺⁺ and Mg⁺⁺ (HBSS⁻⁻), and pH 7.4. Cells were then adjusted at concentration of 1×10⁶ cells/mL by using HBSS, containing Ca⁺⁺ and Mg⁺⁺ (HBSS⁺⁺) for chemiluminescence assay.

NMRI mice of 25 to 30 gm weight were injected aseptically with 1 mL of heat inactivated fetal bovine serum (FBS) in the peritoneum cavity, using sterile 1 mL syringe and mice were kept for further 72 h. Mice were then killed with cervical dislocation and their whole body was sterilized using 70% ethanol. 10 mL of 10% RPMI medium without antibiotic was injected in the peritoneum cavity. Injected media containing macrophages from peritoneal cavity was then collected by cutting the skin of peritoneum cavity from the lower side, with the help of sterile syringe. Collected media was then centrifuged at 4° C. with speed of 150 g for 20 min. Supernatant was discarded and cells pellet was washed using incomplete RPMI at 150 g for 10 minutes at 4° C. Supernatant was discarded and pellet was re-suspended in 1 mL of complete RPMI. Cells were counted and concentration was adjusted to 1×10⁶/mL for chemiluminescence assay.

Luminol enhanced chemiluminescence technique was applied to study the effect of compound on ROS from phagocytes, Using whole blood, neutrophil and macrophages Briefly, 25 μL diluted whole blood (1:50 dilution in sterile HBSS⁺⁺) or 25 μL of PMNs (1×10⁶), or 25 μL of isolated macrophages (1×10⁶) were incubated with 25 μL of compound in triplicates and using three different concentrations (1, 10 and 100 μg/mL) using white 96 wells plates. Wells received HBSS⁺⁺ and cells only without compound served as positive control. 25 μL of serum opsonized zymosan-A (SOZ), followed by 25 μL luminol (7×10⁻⁵ M), was added to each well to maintain a volume of 100 μL/well. Plates were then incubated at 37° C. for 30 minutes in the thermostated chamber of the luminometer. Results were monitored as relative light unit (RLU) reading, with peak and total integral values set with repeated scans at 60 seconds intervals, and 1 second points measuring time.

Heparinized blood was obtained by vein puncture aseptically from healthy volunteers (25-38 years age). Blood (10 mL) was mixed with incomplete RPMI, layered on 5 mL of Lymphocyte separation medium and centrifuged at 400 g for 20 min at room temperature. Buffy layer was collected and mixed with incomplete RPMI and centrifuged at 4° C. for 10 min at 300 g. Cells were obtained after the RBCs (if any) lysis using hypotonic solution, and washed at 4° C. for 10 min at 300 g. The cells were then suspended in complete RPMI containing 5% FBS and their concentration was adjusted to 2×10⁶ cells/mL for T-cell proliferation assay.

Cell proliferation assay was conducted through standard thymidine incorporation technique. Briefly, isolated T-cells from peripheral blood of healthy individuals were plated at a concentration of 2×10⁶ cells/mL in a round bottom 96-well tissue culture plates. Cells were stimulated with 7.5 μg/mL of PHA. Various concentrations of compound in triplicate were added, ranged between 0.3 to 50 μg/mL in order to calculate IC₅₀ value. The plate was then incubated for 72 hours at 37° C. in 5% CO₂ incubator. After 72 hours, cultures were pulsed with 0.5 μCi/well tritiated thymidine (Amersham Pharmacia Biotech), and further incubated for 18 h. Cells were harvested onto a glass fiber filters using cell harvester (Innotech, Dottikon, Switzerland). The level of the thymidine incorporated into the cells was measured by a liquid scintillation counter (LS 6500, Beckman Coulter, Fullerton, Calif., USA). Results were expressed as mean count per minute (CPM). The inhibitory activity of compounds on T-lymphocyte proliferation was calculated using the following formula:

$$Inhibitory activity ( \% ) = \frac{Control group ( CPM ) - Experiment group ( CPM )}{Control group ( CPM )} \times 100$$

Cell cytotoxicity assay was performed using three different cell lines NIH 3T3 (mouse embryo, fibroblast cells), CC1 (rat, epithelial, liver cell) and MDBK (bovine kidney) cells using MTT (3-[4,5-dimethylthiazole-2-yl]-2,5-diphenyl-tetrazolium bromide) colorimetric assay. Briefly 3T3 were grown in DMEM, supplemented with 10% of fetal bovine serum (FBS), and 1% penicillin-streptomycin whereas CC1 (rat epithelial liver cells) were cultured in EMEM, 2 mM Glutamine, 1% Non-Essential Amino Acids (NEAA), 20 mM HEPES, 10% Foetal Bovine Serum (FBS) and 1% penicillin-streptomycin, and MDBK cells were grown in 10% of fetal bovine serum (FBS) in 75 cm² flask, and incubated in 5% CO₂ incubator at 37° C. until they attain 70% confluency. Cells were then harvested by centrifuging at 200 g for 5 min at 25° C., counted using heamocytometer and their concentration was adjusted to 6×10⁴ cells/mL. 100 μL of Cells were then plated in 96 well plate and incubated overnight. Next day the supernatant was carefully removed and 200 μL of fresh medium was added with three different concentration of compound (1, 10, 50) μg/mL. After 48 h incubation, 50 μL of MTT (2 mg/mL) was added to each well and incubated for another 4 h. Later MTT was removed gently and 100 μL of DMSO was added to each well and the plate was incubated at room temperature for 10 minutes with gentle agitation. The extent of MTT reduction to formazan within cells was calculated by measuring the absorbance at 540 nm; using a micro plate reader (Spectra Max plus 340, Molecular Devices, Calif., USA). The cytotoxicity was recorded as concentration causing 50% growth inhibition (IC₅₀). The percent inhibition was calculated by using the following formula:

$$\% inhibition = 100 - \frac{Mean of OD of test compound}{Mean of OD of positive compound} \times 100$$

Briefly THP-1 cells were washed with ice cold PBS, and solubilized by adding 100 μl of 1×cell lysis buffer with 1 mM PMSF. The protein concentrations in the supernatants of lysates were measured by the modified lowery method. The proteins were separated by 12% SDS PAGE and transferred to a nitrocellulose membrane using blotting apparatus. The membranes were soaked in block buffer (3% BSA in PBS) and incubated over night with respective primary antibodies including Rabbit polyclonal to P38 antibody, Rabbit polyclonal to P38 antibody phospho Y182+T180, Anti-NFκB p65 antibody and Anti-NFκB antibody phospho S536 (Abcam, Cambridge, UK), followed by horseradish peroxidase conjugated secondary antibodies (Goat anti rabbit IgG, Thermoscientific, Rockford, USA). The color was developed by staining the nitrocellulose membranes with DAB (dimino-benzidine) stain.

All studies were carried out using Female Sprague Dawley (SD) rats weighing 150-250 gms. Animals were kept at 21±2° C. and on a 12-h light/dark cycle, received standardized pelleted diet and tap water. Experiments were performed under the ethical guidelines of the International Association for the study of pain in conscious animals. Each group contains 12 animals with randomly distributed weights into each treatment group.

Arthritis was induced by injecting freshly prepared 100 μL of 1 mg suspension of lyophilized Mycobacterium tuberculosis H37Ra (MT H37Ra; DIFCO Laboratories, Detroit, Mich., USA), subcutaneously at the rat tail base using sterile hypodermic needle. Inoculation was carried out under anesthesia with a combination of 20 mg/kg ketamine and 5 mg/kg xylazine, injected intraperitoneally. Compound and the reference drug were administered daily, intraperitoneally using sterile syringes. The positive control received vehicle (5% DMSO+PBS) or saline only; treatment groups received test compounds (25 mg/kg), whereas reference group received indomethacin (5 mg/kg). The doses of the test compounds were selected after preliminary dose finding studies. The treatment was started on the same day after vaccination.

The animals were sacrificed at the end of the experiment and samples were taken from the knee joints. Sample fixation were done using 10% formalin following decalcification. The samples were then processed, embedded, cut, mounted and stained with hematoxylin and eosin (H & E) dye for microscopic evaluation.

The quantitative measurement of TNF-α and IL-1β was done in serum of all groups of animals including arthritic and non-arthritic using commercial ELISA assay kits (Abcam, USA) according to the manufacturer's instructions. The samples and standards were all run in duplicate and the data were then averaged.

Peroxide (PO) and nitric oxide (NO), in serum were determined in the serum samples of both the normal and arthritic groups, using quantitative colorimetric assay kits, i.e., the Quantichrom ™ Nitric Oxide Assay Kit, and the Quantichrome™ Peroxide assay kit Diox-250 (BioAssay Systems, Hayward, Calif., USA). The nitric oxide assay system is designed to accurately measure NO production following reduction of nitrate to nitrite (improved Griess method). The samples were analyzed in duplicates using 96-well plate and the plate was read at 540 nm. The Quantichrome™ Peroxide assay kit Diox −250 system is an improved method which utilizes the oxidation of Fe⁺⁺ by peroxide present in the analyzed sample resulting in formation of purple complex that gives the accurate measure of peroxide level in the sample when read at 540-610 nm.

The concentration of reduced form of glutathione was determined in serum using the colorimetric Quantichrome ™ Glutathione assay kit (Bioassay Systems) in duplicates, using 96 well plates and according to manufacturer's instructions. The OD recorded at 412 nm which directly represents the level of glutathione in serum.

Measurement of RA factor in rat serum was determined using Tina-quant RFII Kit (Roche Diagnostics, USA) and automated analyzers according to manufacturer's instructions. The test principle involves the reaction of latex bound heat inactivated IgG (antigen) with RF antibodies in the sample to form Ag/Ab complex resulting in agglutination which can be measured turbidimetrically.

In in vitro studies data was analyzed using student T-test and results were presented as mean SD of mean whereas for in vivo studies the Statistical Package for the Social Sciences (SPSS) software was used to analyze the data. Throughout this study, mean±SEM were used to describe the data. The data were analyzed using one-way analysis of variance (ANOVA). The Bonferroni's post hoc test was used to determine which group mean differs (the value of 0.05 and other values below 0.05 were considered as significant).

What is claimed is:

1. Use of Patuletin or a stereoisomer, or a pharmaceutically acceptable salt, ester, or solvate thereof, in the manufacture of a medicament for the treatment of a disease or disorder, which is mediated by TNF-alpha activity.

2. The use of claim 1, wherein the compound is in association with at least one pharmaceutically acceptable excipient.

3. A method of treating a disease or disorder which is mediated by TNF-alpha activity, the method comprising administering to a subject in need of such treatment an effective amount of Patuletin or a steoisomer, or a pharmaceutically acceptable salt, ester, or solvate thereof.

4. The method of claim 3, wherein the disease or disorder is inflammation.

5. The method of claim 3, wherein the disease or disorder is septic shock.

6. The method of claim 3, wherein the disease or disorder is arthritis.

7. The method of claim 3, wherein the disease or disorder is cancer.

8. The method of claim 3, wherein the disease or disorder is acute respiratory distress syndrome.

9. The method of claim 3, wherein the disease or disorder is an inflammatory disease.

10. The method of claim 9, wherein the inflammatory disease is selected from the group consisting of rheumatoid arthritis, osteoarthritis, inflammatory bowel disease, and asthma.

11. The method of claim 3, wherein the disease or disorder is an autoimmune disorder.

12. The method of claim 11, wherein the autoimmune disorder is selected from the group consisting of diabetes, asthma, and multiple sclerosis.

13. The method of claim 3, wherein the disease or disorder is a disease associated with excess glucocorticoid levels.

14. The method of claim 13, wherein the disease is Cushing's disease.
